(19) United States
(12) Patent Application Publication
(10) Pub. No.: US 2021/0283346 A1
(43) Pub. Date: Sep. 16, 2021

(54) COVER DEVICE FOR AN INHALER

(71) Applicant: KINDEVA DRUG DELIVERY L.P., St. Paul, MN (US)

(72) Inventor: John A. Alimi, Cambridge (GB)

(21) Appl. No.: 17/261,908

(22) PCT Filed: Jul. 24, 2019
(86) PCT No.: PCT/IB2019/056336

§ 371 Date: Jan. 21, 2021

(30) Foreign Application Priority Data
Jul. 25, 2018 (GB) 1812142.6

(51) Int. Cl. A61M 15/00

(52) U.S. Cl. CPC A61M 15/0001; A61M 15/0021; A61M 15/009

(57) ABSTRACT

An inhaler having an air inlet, a medicament source, and an outlet for delivering an air and medicament admixture to a user, wherein the inlet is defined by a coverplate assembly comprised of a plurality of overlapping coverplates to inhibit the ingress of airborne contaminants.

### FIELD

The present application generally relates to medicinal inhalers and, more particularly, to a cover for the air inlet of a medicinal inhaler.

### BACKGROUND

Delivery of aerosolized medicament to the respiratory tract for the treatment of respiratory and other diseases is conventionally done using inhalers of either the pressurised metered dose inhaler (pMDI), the dry powder inhaler (DPI) or the nebulizer type. pMDI inhalers in particular have become an industry standard, and are familiar to many patients who suffer from either asthma or from chronic obstructive pulmonary disease (COPD). Conventional pMDI devices comprise an aluminum canister, sealed with a metering valve, which contains the medicament formulation. Generally, the medicament formulation is a pressurized formulation containing either fine particles of one or more medicinal compounds suspended in a liquefied hydrofluoroalkane (HFA) propellant, or a solution of one or more medicinal compounds dissolved in a propellant/co-solvent system. Formulations incorporating one drug in solution and another one in suspension form are also known.

Dry powder inhalers are often described as “breath-actuated” because many of them utilise the energy of a patient's inhaled breath to release a dose of powdered medicament, usually admixed with a suitable carrier powder, for the patient to inhale directly. However, some DPIs are designed to dispense the dose actively by releasing a separate energy source to the powder, upon activation of a mechanism by the patient's inhalation.

In a conventional pulmonary pMDI, the sealed canister is provided to the patient in an actuator. The actuator is conventionally a generally L-shaped plastic molding comprising a generally cylindrical vertical tube that surrounds the canister plus a generally horizontal tube that forms a patient portion (e.g., a mouthpiece or nosepiece) that defines an inspiration (or inhalation) orifice. To use such an inhaler, the patient exhales, places the patient port into a body cavity (e.g., a mouth or nose) and then inhales to draw air through the inspiration orifice. The majority of such inhalers are of the pulmonary “press-and-breathe” type, where the patient must press down on the protruding end of the canister in order to operate the metering valve to release a metered dose of medicament from the canister into the inhaled air stream and thence through the mouthpiece into their lungs.

To overcome what can be quite a challenge for some patients, pMDI device designs have been created that employ automatic breath-actuated triggering, releasing a dose only in response to the patient's inhaled breath. The AUTOHALER™ metered dose inhaler, available from 3M Company, St. Paul, Minn., USA, and the EASIBREATHE™ inhaler, available from Teva Pharmaceutical Industries Ltd., Israel, are two such pMDI devices that use breath-actuation to attempt to better coordinate dose release with inhalation. Many other inhaler breath-actuated mechanisms have been proposed, but tend to have one or more weaknesses or disadvantages, for example high component counts (and hence high manufacturing costs), complexity (typically giving rise to difficulties of assembly and/or complex dimensional tolerance stack-ups, etc.), performance issues (it is difficult to balance sensitivity (a light triggering force) against stability at rest and/or prior to inhalation) and/or excessive size and/or a less familiar or more awkward overall inhaler shape.

Both conventional and “press and breath” actuators have an inlet which allows the user to draw in ambient air with which the medicament is then entrained within the body of the inhaler. This inlet is often physically protected from the elements to inhibit the ingress of foreign bodies into the airflow. An example of known physical protection is a perforated surface which permits the throughflow of air but inhibits the ingress of objects larger than the individual perforations.

However, this type of inlet cover can choke the air flow into the inhaler due to the small size of the perforations required to inhibit ingress of foreign bodies. Furthermore, the inlet is typically arranged at the top of the inhaler allowing rain to pass into the air flow through the perforations in the event of outside use of the inhaler.

### SUMMARY

According to the present disclosure there is provided an inhaler having an air inlet, a medicament source, and an outlet for delivering an air and medicament admixture to a user, wherein the inlet comprises a coverplate assembly to inhibit the ingress of airborne contaminants.

Advantageously, the coverplate assembly inhibits the ingress of airborne contaminants such as rainwater whilst maintaining sufficient airflow through the inlet to ensure the efficacy of the medicament dose delivered to the user.

The coverplate assembly can comprise a primary coverplate which is curved in both the longitudinal and transverse planes.

Advantageously, this feature of the present disclosure permits the primary coverplate to shed water in a transverse and longitudinal direction which increases the efficiency with which water is shed away from the air inlet and allows the inhaler to accommodate variations in the angle of use without impeding the performance of the coverplate assembly.

The coverplate assembly can comprise a plurality of curved coverplates.

The plurality of curved coverplates can further include a rear coverplate curved in both the longitudinal and transverse plane,

A rear portion of the primary coverplate can overlap a front portion of the rear coverplate.

The plurality of curved coverplates can further include a first front coverplate curved in the transverse plane.

A front portion of the primary coverplate can overlap a rear portion of the first front coverplate.

The plurality of curved coverplates can further include a second front coverplate curved in the transverse plane.

A front portion of the first front coverplate can overlap a rear portion of the second front coverplate.

The plurality of curved coverplates can further include a third front coverplate curved in the transverse plane.

A front portion of the second front coverplate can overlap a rear portion of the third front coverplate.

The plurality of curved coverplates can further include a fourth front coverplate.

A front portion of the third front coverplate can overlap a rear portion of the fourth front coverplate curved in the transverse plane.

The primary coverplate can have a chamfered front edge.

One or more of the, first, second, third and/or fourth coverplates can have a chamfered front edge.

A gap can be defined between the rear coverplate and the first front coverplate, the gap being covered by the primary coverplate.

A front portion of the primary coverplate can be at a greater angle relative to a base of the assembly than is the first front coverplate.

The first front coverplate can be at a greater angle relative to a base of the assembly than is the second front coverplate.

The second front coverplate can be at a greater angle relative to a base of the assembly than is the third front coverplate.

The third front coverplate can beat a greater angle relative to a base of the assembly than is the fourth front coverplate.

Other features and aspects of the present disclosure will become apparent by consideration of the detailed description and accompanying drawings.

### BRIEF DESCRIPTION OF THE DRAWINGS

Embodiments of the present disclosure will now be described, by way of example, with reference to the following drawings, in which:

FIG. 1 is an isometric side view of the inhaler of the present disclosure;

FIG. 2 is a side view of the inhaler of FIG. 1;

FIG. 3 is an isometric view of the coverplate assembly of the inhaler FIG. 1;

FIG. 4 is a rear elevated isometric view of the coverplate assembly of FIG. 3; and

FIG. 5 is a side cross-sectional view of the coverplate assembly of FIG. 3 sectioned along line V-V in FIG. 4.

### DETAILED DESCRIPTION

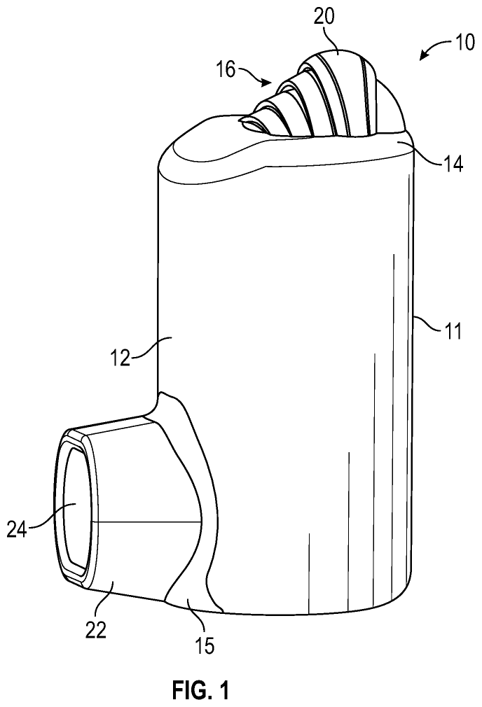

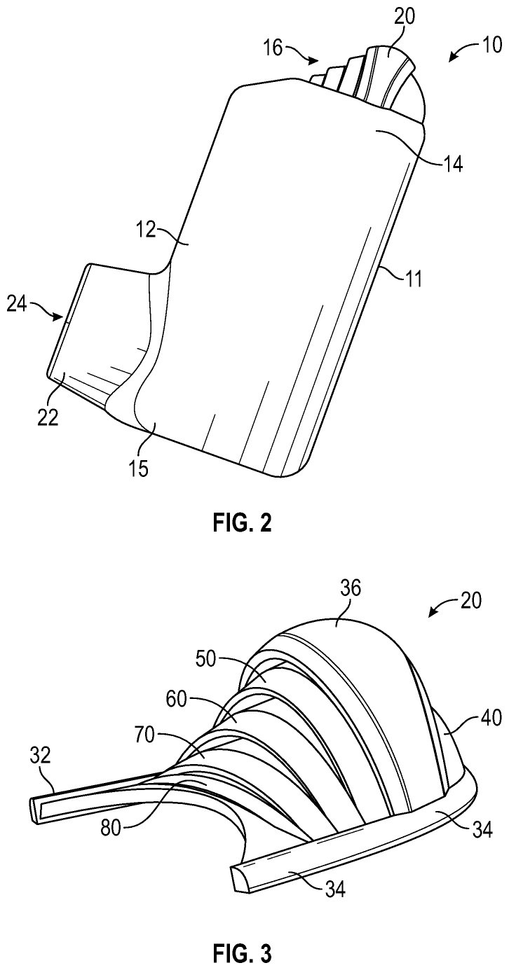

Referring initially to FIG. 1 and FIG. 2, an inhaler in the form of pressurized metered dose inhaler (pMDI) 10 is shown having a chassis 11 which has an upwardly extending main body 12 which houses a medicament source in the form of a canister (not shown for clarity) containing a medicament formulation.

The chassis 11 has a top section 14 and a lower section 15. The top section 14 defines an aperture (not shown) which permits air to enter the chassis 11. Situated atop the aperture is a coverplate assembly 20 which defines an inhaler air inlet 16 in fluid communication with the aperture. The coverplate assembly 20 covers the aperture, such that when viewed from a top of the inhaler in the form of pressurized metered dose inhaler (pMDI) 10 looking down along a line drawn along the axis from the top section 14 through the lower section 15 of the chassis 11, the internal portion of the chassis and the canister cannot be seen. The lower section 15 has a mouthpiece 22 which defines an outlet 24 from the inhaler 10. An air flowpath (not shown for clarity) is defined between the inlet 16 and outlet 24.

The medicament is dispensed from the canister into the air flowpath upon the triggering of the device, such as with a breath actuation mechanism (not shown) in a known manner.

Accordingly, in use, the user draws air into the main body 12 via the air inlet 16. The air is mixed with medicament from the canister, upon the triggering of the breath actuation mechanism in a known manner, to form a medicament admixture. The medicament admixture is then drawn into the mouthpiece and then the mouth by the continued inhalation of the user.

Turning now to FIG. 3, the coverplate assembly 20 can be seen in more detail to have a U-shaped base 30 (see FIG. 5) which has forward facing arms 32, 34 and a curved rear portion 35. The base 30 is affixed to the top section 14 of the inhaler 10 such that the coverplate assembly 20 entirely covers the aperture in the top section 14. Extending between the first and second arms 32, 34 are a series of coverplates. In an embodiment, as depicted in FIG. 3, the series of coverplates includes a top or primary coverplate 36, a rear coverplate 40, a first front coverplate 50, a second front coverplate 60, a third front coverplate 70 and a fourth front coverplate 80. Inhaler air inlets 16 can be formed in between two or more of the overlapping series of coverplates.

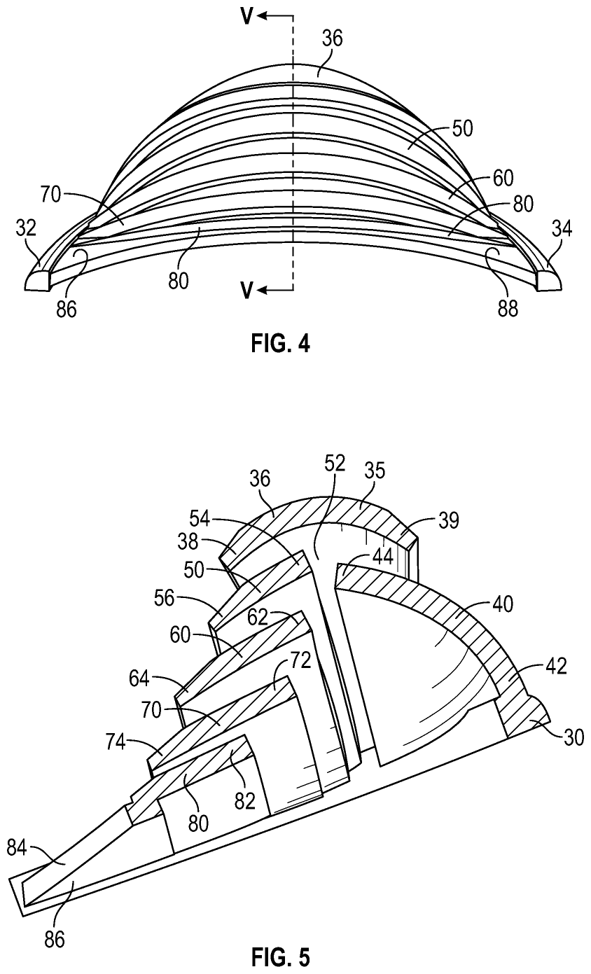

Referring now to FIGS. 4 and 5, a primary coverplate 36 extends between the arms 32, 34 and forms the upper-most part of the inhaler 10 with the inhaler 10 in the upright position as shown in FIG. 1. The primary coverplate 36 is curved in the transverse plane such that it bridges the arms 32, 34. It is also curved in the longitudinal plane (as shown most clearly in FIG. 5) such that its midpoint 35 is higher than a front portion 38 and a rear portion 39. As a result any rain water that lands on the primary coverplate 36 will be shed in all directions, that is to say towards one of the arms 32, 34 and/or towards one, or both, of the front or rear portion 38, 39. The front and rear portions 38, 39 each have a chamfer arranged on their top surface.

Situated rearward of the primary coverplate 36 is a rear coverplate 40. The rear coverplate 40 is shaped approximately in the form of a quadrant, that is to say, the rear half of a hemisphere. The rear coverplate 40 has a rear lower section 42 connected to the base 30 and a front portion 44 which is situated beneath the rear portion 39 of the primary coverplate 36 such that the rear portion 39 of the primary coverplate 36 overlaps the front portion 44 of the rear coverplate 40. In an alternative embodiment (not shown) the rear coverplate 36 may have an air inlet opening between the front portion 44 and the base 30.

The first front coverplate 50 is positioned partially forward of, and partially beneath, the primary coverplate 36 such that the front portion 38 of the primary coverplate 36 overlaps a rear portion 54 of the first front coverplate 50. Like the primary coverplate 36, the first front coverplate 50 is curved in the transverse plane such that it bridges the arms 32, 34. It is positioned relative to the rear coverplate 40 such that there is a gap 52 therebetween (see FIG. 5). The front portion 38 has a chamfer arranged on its top surface.

The second front coverplate 60 is positioned partially forward of, and partially beneath, the first front coverplate 36 such that a front portion 56 of the first front coverplate 50 overlaps a rear portion 62 of the second front coverplate 60. Like the first front coverplate 36, the second front coverplate 60 is curved in the transverse plane such that it bridges the arms 32, 34. The front portion 56 has a chamfer arranged on its top surface.

The third front coverplate 70 is positioned partially forward of, and partially beneath, the second front coverplate 50 such that a front portion 64 of the second front coverplate 60 overlaps a rear portion 72 of the second front coverplate 70. Like the second front coverplate 60, the third front coverplate 70 is curved in the transverse plane such that it bridges the arms 32, 34. The front portion 64 has a chamfer arranged on its top surface.

The fourth front coverplate 80 is positioned partially forward of, and partially beneath, the third front coverplate 70 such that a front portion 74 of the third front coverplate 70 overlaps a rear portion 82 of the fourth front coverplate 80. The fourth front coverplate has a less pronounced transverse curve than do the first, second and third front coverplates 50, 60, 70. The fourth front coverplate 80 has forwardly extending flared portions 86, 88 which are attached to the first and second arms 32, 34 respectively. In embodiments, the overlap between adjacent coverplates can be between about 1 mm and about 5 mm.

The front portion 38 of the primary coverplate 36 is at a greater angle relative to the base 30 than is the first front coverplate 50. The first front coverplate 50 is at a greater angle relative to a base 30 of the assembly 20 than is the second front coverplate 60. The second front coverplate 60 is at a greater angle relative to a base 30 of the assembly 20 than is the third front coverplate 70. The third front coverplate 70 is at a greater angle relative to a base 20 of the assembly than is the fourth front coverplate 80.

Each of the coverplates 36, 40, 50, 60, 70, 80 are separated from the neighboring coverplate so as to leave gaps therebetween that collectively forms the air inlet 16.

In use, the inhaler is held by the user in the upright position as shown in FIG. 1. In the event the inhaler 10 is used outside in conditions of precipitation, particularly rain, the coverplate assembly 20 acts to inhibit the ingress of rain into the body of the inhaler as follows. Rain landing to the left of the centerline V-V shown in FIG. 4 is shed towards the first arm 32. Rain landing to the right of the centerline V-V shown in FIG. 4 is shed towards the second arm 34. Rain landing on the front portion 38 of the primary cover plate is shed forwards under the assistance of the chamfer onto the front portion 56 of the first front coverplate 50. The rain continues to be shed forwards onto successive coverplates until it is deposited safely onto the chassis 11 of the inhaler 10 away from the air inlet 16 and aperture. Similarly, rain landing on the rear portion 39 of the primary cover plate is shed rearwards under the assistance of the chamfer onto the rear coverplate 40 being deposited safely onto the chassis 11 of the inhaler 10.

Accordingly, the coverplate assembly 20 is able to inhibit the ingress of rain water into the body of the inhaler 10 whilst maintaining the requisite airflow through the air inlet 16.

In one embodiment the inhaler comprises electronic components, such as circuit boards, pressure sensors, accelerometers, electronic dose counters, batteries, solenoids, and the like, for which it is important to protect from environmental damage. Examples of suitable electronic inhalers include those described in International Patent Application Publication Nos. WO 2018/200431, WO2017/178764, U.S. Pat. No. 9,782,551, and U. S. Patent Application Publication No. 2016/0051776.

1. An inhaler having an air inlet, a medicament source, and an outlet for delivering an air and medicament admixture to a user, wherein the inlet is defined by a coverplate assembly comprised of a plurality of overlapping coverplates to inhibit the ingress of airborne contaminants, the coverplate assembly including one or more inlet portions formed between the plurality of overlapping coverplates to define the inlet.

2. The inhaler of claim 1, wherein the coverplate assembly comprises a primary coverplate which is curved in both the longitudinal and transverse planes.

3. The inhaler of claim 2, wherein one or more of the plurality of overlapping coverplates are curved.

4. The inhaler of claim 3, wherein the plurality of curved coverplates further includes a rear coverplate curved in both the longitudinal and transverse plane.

5. The inhaler of claim 4, wherein a rear portion of the primary coverplate overlaps a front portion of the rear coverplate.

6. The inhaler of claim 5, wherein the plurality of curved coverplates further includes a first front coverplate curved in the transverse plane.

7. The inhaler of claim 6, wherein a front portion of the primary coverplate overlaps a rear portion of the first front coverplate.

8. The inhaler of claim 6, wherein the plurality of curved coverplates further includes a second front coverplate curved in the transverse plane.

9. The inhaler of claim 8, wherein a front portion of the first front coverplate overlaps a rear portion of the second front coverplate.

10. The inhaler of claim 8, wherein the plurality of curved coverplates further includes a third front coverplate curved in the transverse plane.

11. The inhaler of claim 10, wherein a front portion of the second front coverplate overlaps a rear portion of the third front coverplate.

12. The inhaler of claim 10, wherein the plurality of curved coverplates further includes a fourth front coverplate.

13. The inhaler of claim 12, wherein a front portion of the third front coverplate overlaps a rear portion of the fourth front coverplate curved in the transverse plane.

14. The inhaler of claim 2, wherein the primary coverplate has a chamfered front edge.

15. The inhaler of claim 4 wherein any one or more of the, first, second, third and/or fourth coverplates has a chamfered front edge.

16. The inhaler of claim 6 wherein a gap is defined between the rear coverplate and the first front coverplate, the gap being covered by the primary coverplate.

17. The inhaler of claim 6 wherein a front portion of the primary coverplate is at a greater angle relative to a base of the assembly than is the first front coverplate.

18. The inhaler of claim 8 wherein the first front coverplate is at a greater angle relative to a base of the assembly than is the second front coverplate.

19. The inhaler of claim 10 wherein the second front coverplate is at a greater angle relative to a base of the assembly than is the third front coverplate.

20. The inhaler of claim 12 wherein the third front coverplate is at a greater angle relative to a base of the assembly than is the fourth front coverplate.
